(19) United States
(12) Patent Application Publication
(10) Pub. No.: US 2004/0102830 A1
(43) Pub. Date: May 27, 2004

(54) System for coupling an implanatable medical device to an epicardial site

(76) Inventor: Terrell M. Williams, Brooklyn Park, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/302,290

(22) Filed: Nov. 22, 2002

(51) Int. Cl. A61N 1/05

(52) U.S. Cl. 607/125

(57) ABSTRACT

A device for delivering an implantable medical device to a target site in a heart along a predetermined pathway that includes a generally straight first portion extending from a proximal end to a distal end and a curved second portion extending from the distal end of the first portion to a distal end of the device. The curved second portion includes a first curve portion formed in a first plane and a second curved portion formed in a second plane substantially orthogonal to the first plane to direct the implantable medical device toward an epicardial surface of the heart directly adjacent to the predetermined pathway

### CROSS-REFERENCE TO RELATED APPLICATION

Cross-reference is hereby made to commonly assigned related U.S. application Ser. No. ______ to Matthew D. Bonner et al, filed concurrently herewith, entitled “MULTIPLE BEND CATHETER FOR DELIVERING A LEAD TO A HEART” (Attorney Docket No. P-9297.00).

### FIELD OF THE INVENTION

The present invention generally relates to implantable medical devices, and, more particularly, the present invention relates to a system used to couple a medical device to an epicardial site.

### BACKGROUND OF THE INVENTION

In pacemaker technology and related arts, a pacemaker is implanted into a patient and coupled to a patient's heart with one or more pacing leads that deliver both electrical signals from the heart to the pacemaker, and electrical stimulation from the pacemaker to the heart, to provide therapy that restores function of the heart for proper blood circulation. A pacing lead is usually constructed having an outer polymeric sheath encasing one or more electrical conductors. The conductors may be arranged coaxially or co-linearly and are insulated from one another. A distal end of each conductor is coupled to an electrode and a proximal end of each conductor is coupled to a connector that is insertably coupled to electrical circuitry of the pacemaker. The lead may be introduced into the heart, and a distal end of the lead attached to various sites within the heart, by a variety of techniques. For example, transvenous leads are introduced into the heart by means of a percutaneous introducer sheath inserted into a venous system at a cephalic or subclavian vein. The transvenous lead may be directed to a site in the heart using a stylet that is inserted into a lumen formed within the lead, or using a guide catheter that forms a pathway to the site with a lumen through which the lead may travel. The distal end of the lead may be attached to the heart using a hook or a tined structure. Traditional attachment sites include endocardial surfaces of a right atrium (RA) or a right ventricle (RV). The connector at the proximal end of the lead is ultimately mated with the pacemaker to complete the coupling.

More recently, for the treatment of heart failure, transvenous pacing leads have been directed to the left side of the heart through a coronary venous system that includes a coronary sinus (CS), and coronary veins branching from the coronary sinus. Typically, a guide catheter is used to gain access to the coronary sinus ostium (CS Os), from the RA, by inserting a distal end of the guide catheter into the CS Os. Such an access procedure is commonly referred to as cannulation of the CS Os. As a result, the guide catheter is generally formed in order to reduce the difficulty experienced when cannulating the coronary sinus ostium A distal end of the pacing lead is then inserted through a lumen of the guide catheter and out the distal end of the guide catheter, which remains near the CS Os within the CS. The distal end of the lead is directed outward from the distal end of the guide catheter and through the coronary venous system, to a target site on the epicardial surface of the left ventricle (LV) using either a guide wire or a stylet, positioned within a lumen of the lead.

A lead lumen designed to accommodate insertion of the stylet within the lead extends from a proximal opening at the proximal end of the lead to a distal tip of the lead. The lumen may or may not be open at the distal tip. The stylet is inserted within the lumen of the lead to provide stiffness for advancing the lead forward out of the guide catheter into the coronary venous system. A distal end of the stylet may be shaped to impose a curve on the distal end of the lead, giving direction to the distal tip of the lead.

In the same way, a lead lumen designed to accommodate insertion of the guide wire within the lead extends from a proximal opening at the proximal end of the lead to a distal tip of the lead. However, it is necessary that the lumen also include a distal opening at the distal tip of the lead so that a distal end of the guide wire may be advanced outward from the opening and extended beyond the distal tip of the lead. The distal end of the guide wire is terminated in a relatively soft and formable tip that is steered beyond the distal tip of the lead in order to direct the distal tip of the lead to the target site.

In summary, a typical method for delivering a transvenous pacing lead to the left side of the heart employs a guide catheter to provide a pathway for a lead to the CS, and either a stylet or a guide wire, within the lumen of the lead, to assist in traversing the lead outward from the guide catheter and further through the coronary venous system, which can be tortuous, in order to position the lead tip at the target site on the epicardial surface of the LV. One disadvantage to the lead having a lumen is embodied in an increased diameter of the lead, since a lead having no lumen may have a reduced diameter if space for a lumen is alternatively used for conductors or insulation.

Furthermore, typical transvenous pacing leads introduced into the coronary venous system do not have a means for direct attachment of the lead distal tip to the target site on the epicardial surface of the LV. Frictional forces imposed by the surrounding veins are relied upon to hold the lead tip at the target site. However, these frictional forces tend to be most adequate if an outer diameter of the distal end of the lead is approximately equal to an inner diameter of the surrounding veins so that the distal end of the lead fits snugly in the veins.

At the same time, some leads known in the art have preformed bends along the distal end of the lead to assist in holding the lead tip in position at the target site. However, one disadvantage to using a preformed bend to fixedly position the lead in the vein is a reduction in area available within the veins for blood flow.

Therefore, was is needed is a system by which a medical device may be directed to a target site along the epicardial surface of the LV with a reduced diameter pacing lead that may be more easily fixedly positioned along the epicardial surface.

### SUMMARY OF THE INVENTION

The present invention is directed to a device for delivering an implantable medical device to a target site in a heart along a predetermined pathway that includes a generally straight first portion extending from a proximal end to a distal end and a curved second portion extending from the distal end of the first portion to a distal end of the device. The curved second portion including a first curve portion formed in a first plane and a second curved portion formed in a second plane substantially orthogonal to the first plane to direct the implantable medical device toward an epicardial surface of the heart directly adjacent to the predetermined pathway.

According to an embodiment of the present invention, a device for delivering an implantable medical device to a target site in a heart along a predetermined pathway through the coronary sinus includes a generally straight first portion extending from a proximal end to a distal end and a curved second portion extending from the distal end of the first portion to a distal end of the device. The curved second portion includes a first curve portion formed in a first plane and a second curved portion formed in a second plane substantially orthogonal to the first plane to direct the implantable medical device toward an epicardial surface of the heart directly adjacent to the predetermined pathway. The curved second portion includes a first section, a second section and a third section extending between the first section and the second section, and the first section is positioned at a first angle from the third section in the first plane and the second section is positioned at a second angle from the generally straight first portion in the second plane, the first angle being approximately equal to 90 degrees and the second angle being approximately equal to 30 degrees. The curved second portion has a radius of curvature between approximately 1.5 inches and 2.0 inches.

According to yet another embodiment of the present invention, a system for delivering an implantable medical device to a target site in a heart along a predetermined pathway through the coronary sinus includes a delivery catheter having a generally straight first portion extending from a first proximal end to a first distal end and a curved second portion extending from the first distal end to a distal end of the delivery catheter. A therapy delivery device, slideably receivable within the delivery catheter, extends from a second proximal end to a second distal end. The curved second portion includes a first curve portion formed in a first plane and a second curved portion formed in a second plane substantially orthogonal to the first plane to direct the therapy delivery device outward from the distal end of the delivery catheter toward an epicardial surface of the heart directly adjacent to the coronary sinus.

According to a still further embodiment of the present invention, a fixation element is coupled to the distal end of the therapy delivery device to fixedly engage the therapy delivery device to the epicardial surface. The fixation element includes a tip portion approximately flat along a direction of a central axis of the fixation element and facing inward toward the central axis.

### BRIEF DESCRIPTION OF THE DRAWINGS

These and other advantages and features of the present invention will be more readily understood from the following detailed description of the preferred embodiments thereof, when considered in conjunction with the drawings, in which like reference numerals indicate identical structures throughout the several views, and wherein:

FIG. 1 is a schematic diagram of a heart from an anterior perspective illustrating a coronary venous system about an epicardial surface, including dashed lines depicting a portion of coronary venous system on an opposite, posterior surface of the heart;

FIG. 2 is a plan view having a partial section view illustrating a pacing lead according to the present invention;

FIG. 3A is a side plan view of a delivery catheter according to the present invention;

FIG. 3B is a front plan view of the delivery catheter of FIG. 3;

FIG. 4 is an enlarged side plan view of a delivery catheter shaft according to the present invention;

FIG. 5A is an enlarged front plan view of a delivery catheter shaft according to the present invention;

FIG. 5B is an enlarged front plan view of a delivery catheter shaft according to an alternate embodiment of the present invention;

FIG. 6 is a schematic diagram of a delivery catheter shaft according to the present invention positioned within a coronary sinus of a heart;

FIG. 7 is a sectional view taken along section line A-A of FIG. 6 illustrating a system for coupling a medical device to an epicardial site using a delivery catheter according to the present invention;

FIG. 8 is a sectional view taken along section line B-B of FIG. 6 illustrating a system for coupling a medical device to an epicardial site using a delivery catheter according to an alternate embodiment of the present invention;

FIG. 9A is a side view of a helix fixation element in a system for coupling a medical device to an epicardial site using a delivery catheter according to the present invention; and

FIG. 9B is a top view of the helix fixation element of FIG. 9A

### DETAILED DESCRIPTION OF THE INVENTION

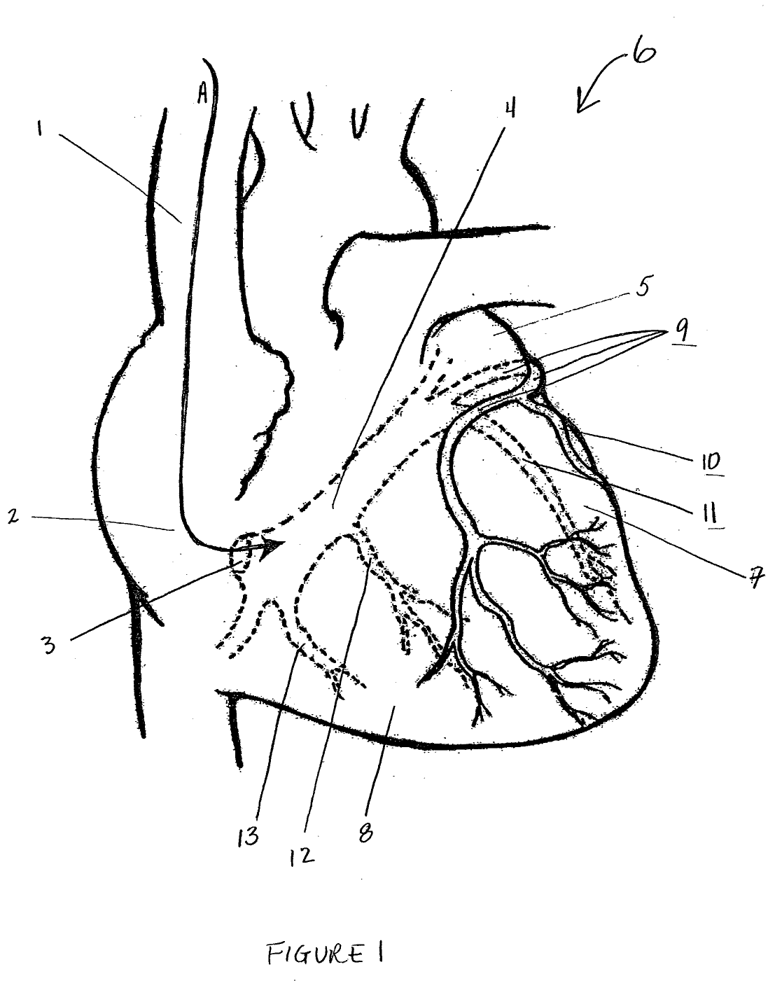

FIG. 1 is a schematic diagram of a heart from an anterior perspective illustrating a coronary venous system about an epicardial surface, including dashed lines depicting a portion of coronary venous system on an opposite, posterior surface of the heart. As illustrated in FIG. 1, a coronary venous system of a heart 6 includes a coronary sinus (CS) 4, along with a middle cardiac vein (MCV) 13, a posterior cardiac vein (PCV) 12, a posterior-lateral cardiac vein (PLV) 11, a great cardiac vein (GCV) 9, and a lateral cardiac vein (LCV) 10 all branching from the coronary sinus 4. In addition, FIG. 1 illustrates a pathway, defined by arrow ‘A’, which may be followed in order to place a pacing lead within coronary sinus 4, extending from a venous access site (not shown) through a superior vena cava (SVC) 1 into a right atrium (RA) 2 of heart 6 and from the right atrium 2 into the coronary sinus 4 through a coronary sinus ostium (CS Os) 3. Pacing leads are typically placed in coronary venous system and coupled to a medical device in order to sense and stimulate a left ventricle (LV) 7 in patients suffering from heart failure, for example.

A first pacing lead stimulating left ventricle 7 typically functions in conjunction with a second pacing lead positioned within and stimulating right ventricle (RV) 8 to provide synchronous activation of right ventricle 8 and left ventricle 7 in order to improve the hemodynamic output of the heart. Synchronous activation is best achieved when first pacing lead stimulates left ventricle 7 at a late activated region of left ventricle 7. Locations in posterior-lateral cardiac vein 11, lateral cardiac vein 10, great cardiac vein 9, or coronary sinus 4, near a junction with great cardiac vein 9, correspond with late activated regions of left ventricle 7 and potential target sites for stimulation of left ventricle 7.

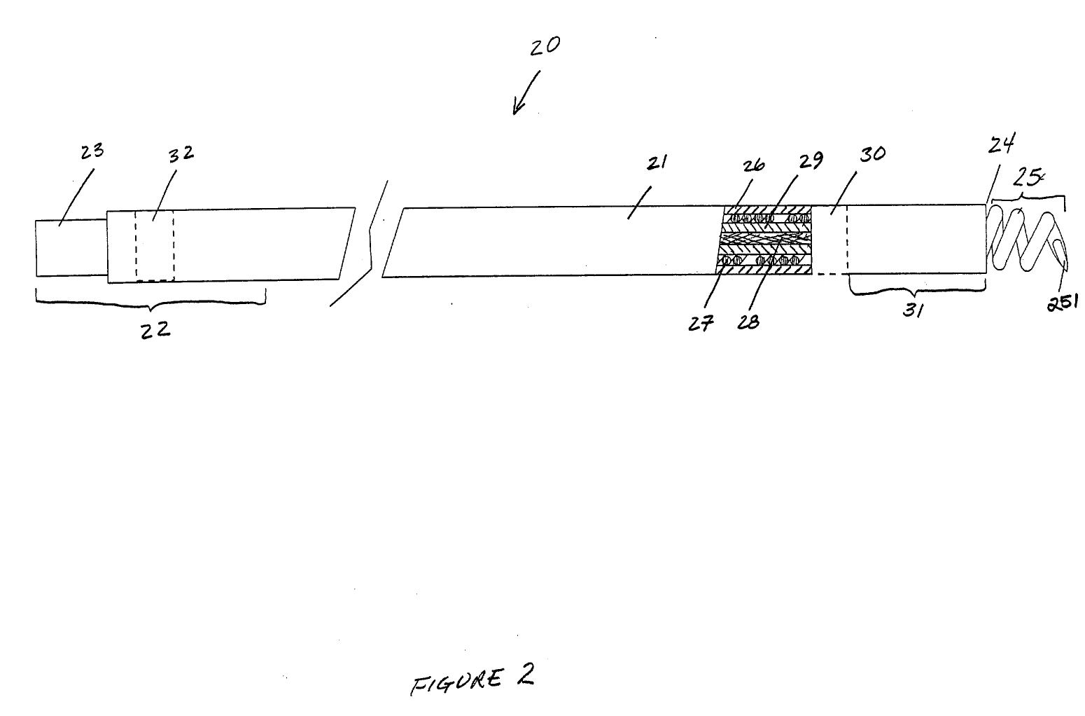

FIG. 2 is a plan view having a partial section view illustrating a pacing lead according to the present invention. As illustrated in FIG. 2, a pacing lead 20 includes an elongated lead body 21 having a proximal portion 22 and a distal end 24. Proximal portion 22, includes a connector pin 23 insertable within a connector block of a pacemaker (not shown), for example. A helix fixation element 25 having a piercing tip 251 extends outward from distal end 24 of lead 20. Lead body 21 is constructed having an outer sheath 26 encasing a coil 27, which is disposed coaxially about an inner member 28 disposed within an inner sheath 29. Pacing lead 20 is essentially isodiametric along its length, with an outer diameter of lead body 21 between approximately 0.025 inches and 0.045 inches. Since lead body 21 does not include an inner lumen, outer diameter of lead body 21 is reduced. Connector pin 23 is adapted for electrical and mechanical coupling with a medical device, and fixation element 25 is adapted to be screwed into a heart wall, as described below, by rotation of lead body 21 when piercing tip 251 is positioned within coronary sinus 4 and becomes engaged along an epicardial surface of heart 6. When fixation element 25 functions as an electrode, as in alternate embodiments described below, Fixation element 25 is preferably formed of a platinum iridium alloy, although it is understood that other biocompatible and biostable materials may also be used, including but not limited to such materials as palladium, titanium, tantalum, rhodium, carbon, vitreous carbon and alloys, oxides and nitrides of such metals or other conductive or even semi-conductive materials well known in the art.

In a first unipolar embodiment of pacing lead 20, outer sheath 26 is an insulative element and coil 27 is a conductive element, coupled, at a proximal end, to connector pin 23 and, at a distal end, to helix fixation element 25, which doubles as an electrode, and inner member 28 is a structural element. Outer sheath 26 is formed of either a silicone rubber or polyurethane, well known in the art, or any other flexible, biostable and biocompatible insulative polymer material. Coil 27 is formed of single or multiple wire filars made of MP35-N alloy, well known in the art, or any other biostable and biocompatible material that is capable of reliably conducting electrical current after having been subjected to numerous, repeated bending and torsional stresses. Inner member 28, which may or may not be conductive, is coupled to connector pin 23 and fixation element 25 to provide tensile strength to lead body 21. Inner member 28 is a cable, for example, formed from synthetic filaments or metallic wires, and inner sheath 29 is a biostable and biocompatible flexible polymer coating or tube encasing inner member 28 in order protect inner member 28 from mechanical stresses or hydrolytic degradation. Alternate inner constructions providing tensile strength for a lead body are disclosed in U.S. Pat. No. 5,246,014 issued to Williams et al. and co-pending U.S. patent application Ser. No. 09/559,161 to Williams and Chivers, both incorporated herein by reference in their entireties. Coupling of coil 27 to both connector pin 23 and fixation element 25 is primarily electrical, while coupling of inner member 28 to both connector pin 23 and fixation element 25 is primarily mechanical. Couplings of coil 27 with connector pin 23 and fixation element 25 are formed with either a weld or a crimp such as is commonly used in the art.

In an alternate unipolar embodiment of pacing lead 20, inner member 28 is a conductive element coupled, at a proximal end, to connector pin 23 and, at a distal end, to helix fixation element 25, which doubles as an electrode. Inner sheath 29 is an insulative element for first inner member 28, and coil 27 acts only as a structural element to provide torsional stiffness to lead body 21. Inner member 28 is preferably a cable formed from wires made of MP35-N alloy, well known in the art, or any other biostable and biocompatible material that is capable of reliably conducting electrical current after having been subjected to numerous, repeated bending and torsional stresses. Inner sheath 29 is formed of a silicone rubber, a polyurethane, or a fluoropolymer, all insulative materials known in the art, or any other flexible, biostable and biocompatible insulating material. Coil 27 is formed of any biostable and biocompatible material that is sufficiently stiff to provide adequate torque transfer from proximal portion 22 of lead 20 to fixation element 25 at distal end 24 of lead 20. Coupling of inner member 28 to both connector pin 23 and fixation element 25 is performed using a crimp or a weld, such as are well known in the art, and must be both mechanically and electrically stable, while coupling of coil 27 to proximal portion 22 and fixation element 25 is primarily mechanical (in order to transfer torque from proximal portion 22 to fixation element 25). Coil 27 may be coupled by crimps or welds or embedded in inner sheath 29 along a length from proximal portion 22 to distal end 24.

In an alternate unipolar embodiment, helix fixation element 25 does not function as an electrode. Rather, a ring electrode 30 (show with dashed lines) is incorporated coaxially about a distal portion of lead body 21 and is coupled to coil 27 that is a conductive element. As in first unipolar embodiment, coupling of coil 27 to both connector pin 23 and fixation element 25 is primarily electrical, while coupling of inner member 28 to both connector pin 23 and fixation element 25 is primarily mechanical. Couplings are formed with either a weld or a crimp such as is commonly used in the art. A spacing 31 between ring electrode 30 and helix fixation element 25 is less than approximately 0.02 inches, in order to locate electrode ring 30 close enough to a fixation site for tissue contact, while fixation element 25 is isolated from both ring electrode 30 and coil 27. Ring electrode 30 is preferably formed of a platinum alloy but other materials may also be used, including but not limited to such materials as palladium, titanium, tantalum, rhodium, iridium, carbon, vitreous carbon and alloys, oxides and nitrides of such metals or other conductive or even semi-conductive materials. Of course, some materials are incompatible with others and may not be effectively used together. The limitations of specific materials for use with others are well known in the art.

Alternatively, in a bipolar embodiment of pacing lead 20, both coil 27 and inner member 28 are conductive, and therefore inner sheath 29 is an insulator between coil 27 and inner member 28. In this embodiment coil 27 is coupled, at a proximal end, to a connector ring 32 (shown with dashed lines) and, at a distal end, to ring electrode 30, and inner member 28 is coupled, at a proximal end, to connector pin 23 and, at a distal end, to helix fixation element 25, which doubles as an electrode. Spacing 31 between ring electrode 30 and helix fixation element 25 is between approximately 0.2 inches and 0.4 inches, a range well known in the pacing art for inter-electrode spacing. Elements of bipolar embodiment are similar to those of aforementioned unipolar embodiments.

A means for steroid elution may be incorporated into any of the aforementioned embodiments of pacing lead 20 near distal end 24. Such steroid elution means may take the form of a monolithic controlled release device (MCRD), preferably constructed from silicone rubber and loaded with a derivative of dexamethasone, such as the water-soluble steroid dexamethasone sodium phosphate. MCRD construction and methods of fabrication are found in Stokes, U.S. Pat. No. 4,506,680 and related U.S. Pat. Nos. 4,577,642, 4,606,118, and 4,711,251, which are incorporated in their entirety herein. Alternatively a steroid coating containing a no more than sparingly water-soluble steroid such as beclomethasone diproprionate or dexamethasone acetate may be applied to surfaces of electrode ring 30 and/or helix fixation element 25. The steroid coating may be applied directly to surfaces or portions of surfaces preserving structural integrity of electrode ring 30 and/or fixation element 25 and taking up less space than an MCRD. A preferred embodiment of the present invention includes the steroid coating on fixation element 25. A steroid coating composition and method of application found in Williams, U.S. Pat. No. 5,987,746, which is incorporated in its entirety herein.

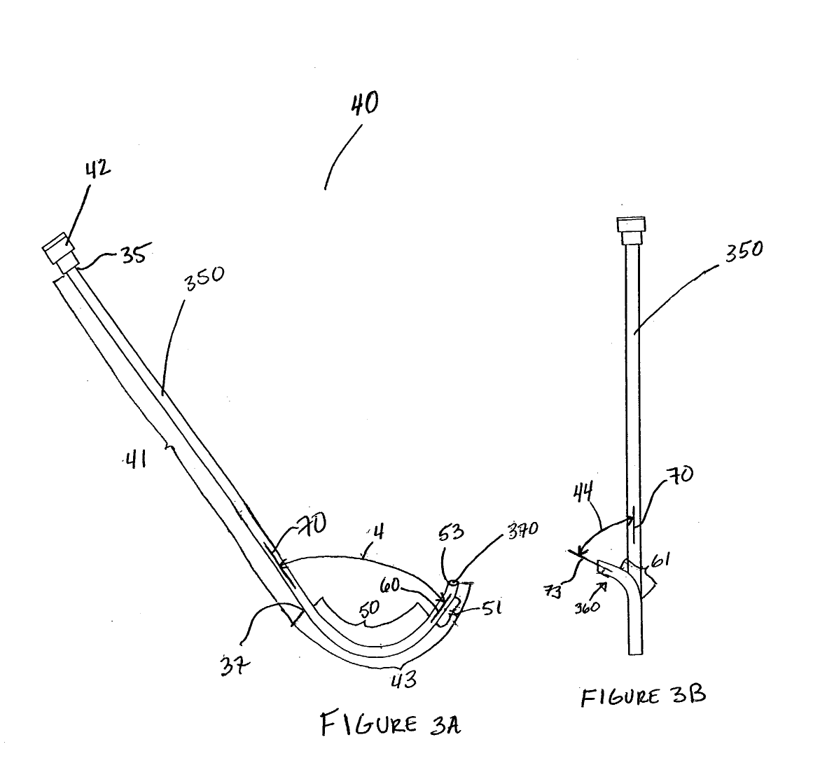

FIG. 3A is a side plan view of a delivery catheter according to the present invention. FIG. 3B is a front plan view of the delivery catheter of FIG. 3A. As illustrated in FIGS. 3A and 3B, a delivery catheter 40 according to the present invention includes an elongated shaft 350 having an inner lumen 370 that extends through shaft 350, which slideably receives pacing lead 20, and a hub 42 positioned at a proximal end 35 of shaft 350. Shaft 350 includes a generally straight proximal portion 41 that extends from proximal end 35 to a distal end 37. Distal portion 43, which extends distally from distal end 37 of proximal portion 41 to a distal end 53 of shaft 350, includes an orientation curve portion 50, a distal tip portion 360 and a transition zone portion 51 extending between orientation curve portion 50 and distal tip portion 360.

According to the present invention, an outer diameter of delivery catheter shaft 350, which is between approximately 0.05 inches and 0.07 inches, is small enough so that deliver catheter shaft 350 slideably passes through a standard guide catheter having an outer diameter between approximately 0.09 inches and 0.120 inches, and into the coronary venous system through coronary sinus 4. Delivery catheter shaft 350 is formed of a biocompatible polymer such as polyethylene, polyester, polyurethane, or a fluoropolymer, or a combination of polymers, a variety of constructions of which are well known in the art.

According to the present invention, as illustrated in FIGS. 3A and 3B, in order for distal tip portion 360 of delivery catheter shaft 350 to be directed toward the heart 6 when positioned in coronary venous system, orientation curve portion 50 and transition zone potion 51 of distal portion 43 of shaft 350 form a first curved portion of distal portion 43 in a first plane corresponding to the plane of page containing FIGS. 3A and 3B, while distal tip portion 360 includes a distal curve 61 in order to form a second curved portion of distal portion 43 in a second plane approximately orthogonal to the first plane, i.e., extending out of the page containing FIGS. 3A and 3B. A central axis 60 of delivery catheter shaft 350 along transition zone 51 between orientation curve 50 and distal tip portion 360 is at an angle 45 of approximately 90 degrees from an a central axis 70 of delivery catheter shaft 350 along proximal portion 41, and a central axis 73 of delivery catheter body 350 along distal tip portion 360 is at an angle 44 of approximately 30 degrees from axis 70 of proximal portion 41. According to the present invention, an axial length of proximal portion 41 is between approximately 10 inches and 15 inches, an axial length of orientation curve 50 is between approximately 3 inches and 5 inches, an axial length of transition zone 51 is between approximately 1 inch and 2 inches, an axial length of distal curve 61 is between approximately 0.4 inches and 0.5 inches, and an axial length of distal tip portion 360 is between approximately 0.15 inches and 0.2 inches.

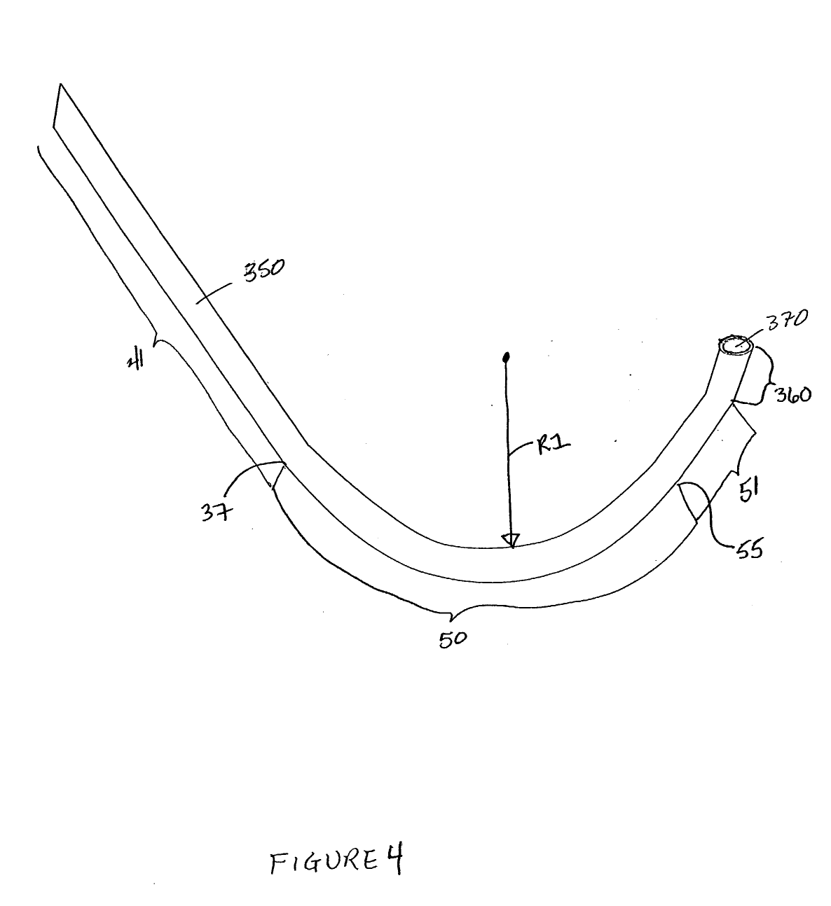

FIG. 4 is an enlarged side plan view of a delivery catheter shaft according to the present invention. As illustrated in FIG. 4, delivery catheter shaft 350 according to the present invention includes an orientation curve 50 extending from distal end 37 of proximal portion 41 to a proximal end 55 of a transition zone 51. In a preferred embodiment, orientation curve has a radius R1 of between approximately 1.5 inches and 2 inches.

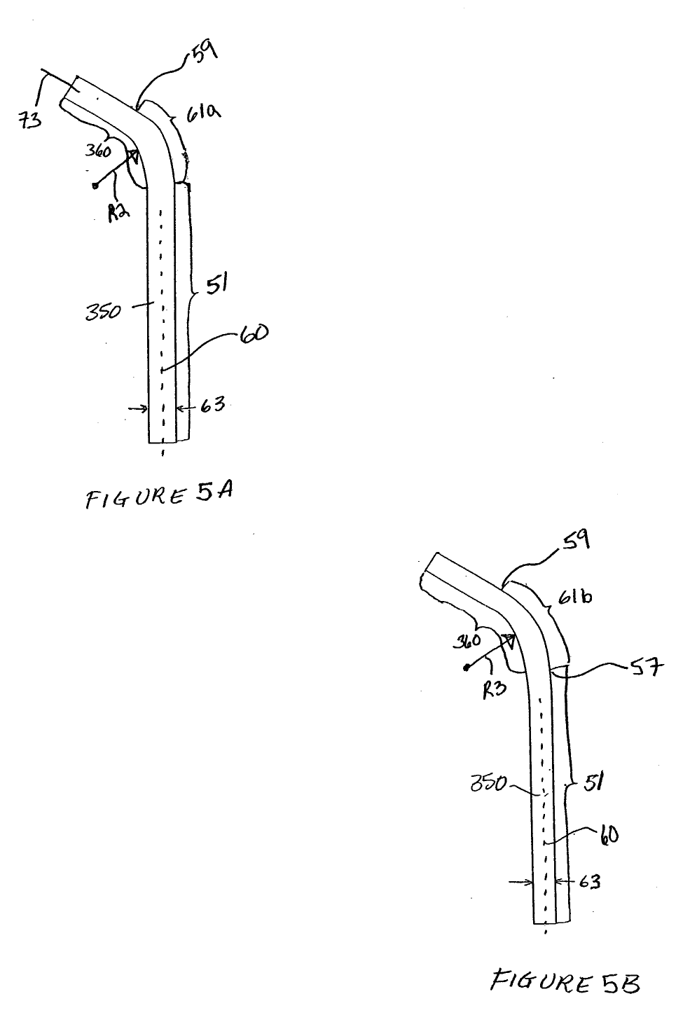

FIG. 5A is an enlarged front plan view of a delivery catheter shaft according to the present invention. As illustrated in FIG. 5A, catheter delivery shaft 350 includes a distal curve 61 a extending from a distal end of transition zone 51 to a proximal end of distal tip portion 360. According to a preferred embodiment of the present invention, a radius R2 of distal curve 61a is between approximately 0.35 inches and 0.45 inches and an outer diameter 63 of delivery catheter shaft 350 is between approximately 0.06 inches and 0.07 inches.

FIG. 5B is an enlarged front plan view of a delivery catheter shaft according to an alternate embodiment of the present invention. As illustrated in FIG. 5B, according to an alternate embodiment of the present invention, catheter delivery shaft 350 includes a distal curve 61b extending from a distal end of transition zone 51 to a proximal end of distal tip portion 360 having a radius R3 between approximately 0.45 inches and 0.6 inches, and an outer diameter 64 of delivery catheter shaft 350 is between approximately 0.05 inches and 0.06 inches.

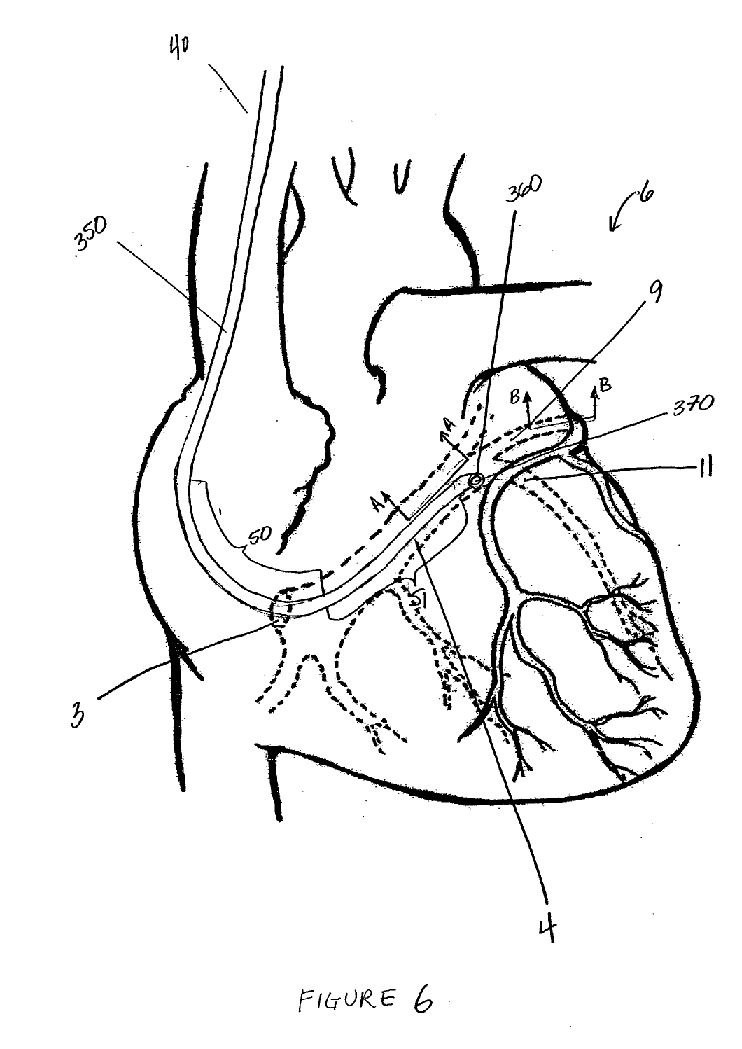

FIG. 6 is a schematic diagram of a delivery catheter shaft according to the present invention positioned within a coronary sinus of a heart. Sections A-A and B-B of FIG. 6 correspond to examples of alternate locations in coronary venous system for navigation of delivery catheter 40. A mapping electrode (not shown) may be coupled to distal tip portion 360 of delivery catheter 40 to provide a means for selecting a target site. As illustrated in FIG. 6, according to the present invention, a first curved portion is formed by orientation curve portion 50 and transition zone portion 51 in a plane corresponding to the plane of the page containing FIG. 6, and a second curved portion formed at distal tip portion 360 of delivery catheter shaft 350 is oriented to direct distal tip portion 360 toward epicardial surface, in a plane substantially orthogonal to the plane corresponding to the first curved portion, i.e., out of the page, in order to direct piercing tip 251 of helix fixation element 25 toward a target implant site on the epicardial surface (recalling that coronary venous system shown in hashed lines in FIG. 6, including coronary sinus 4, is located along the posterior surface of heart 6).

Delivery catheter shaft 350 may be maneuvered independently into coronary sinus 4 from a venous access site (not shown), or could be passed through a standard guide catheter (not shown) whose tip has cannulated coronary sinus ostium 3, or passed over a guide wire (not shown) whose tip has cannulated coronary sinus ostium 3. According to the present invention, delivery catheter 40 provides a complete path for inserting pacing lead 20 at a venous access point and extending lead 20 to a target site. Without delivery catheter 40, chronic advantages of pacing lead 20 having a reduced diameter and helix fixation element 25, such as preservation of venous hemodynamics and implant stability, become acute disadvantages in terms of delivering pacing lead 20 to target implant site. For example, even if a standard guide catheter, having distal tip positioned inside coronary sinus ostium 3 is provided as a partial path for pacing lead 20, it would be almost impossible to advance a relatively small lead body 21 of pacing lead 20 to target site without help from a stylet or a guide wire, since lead body 21 lacks stiffness and direction for maneuvering. Additionally, exposed piercing tip 251 of helix fixation element 25 would tend to catch on sites in the venous system prior to arriving at target implant site and, once there, would have no means of direction toward epicardial surface for attachment. Therefore, delivery catheter 350 lends stiffness to lead body 21 and forms both a temporary shroud for piercing tip 251 and a path to direct piercing tip 251.

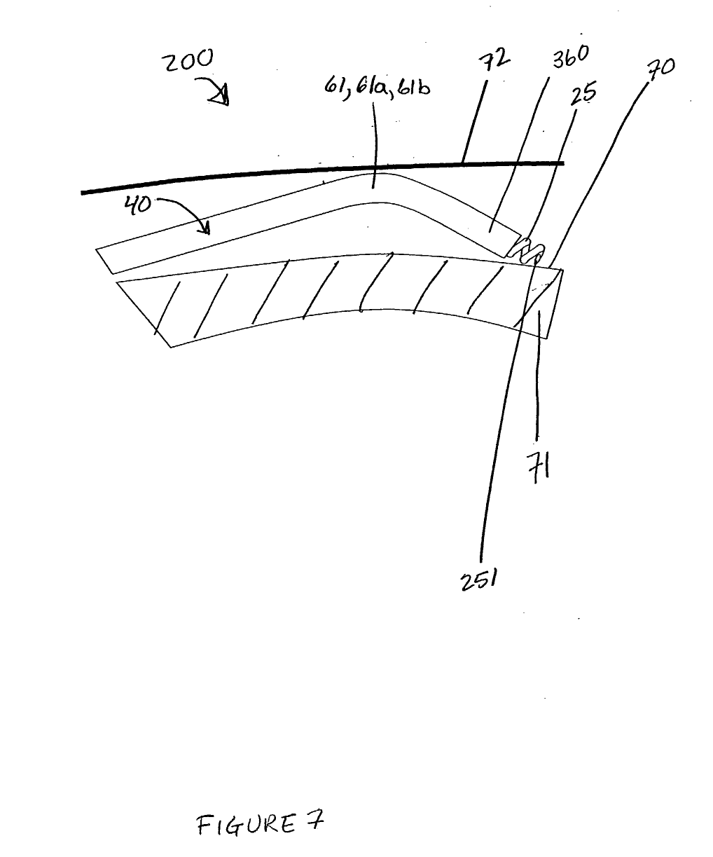

FIG. 7 is a sectional view taken along section line A-A of FIG. 6 illustrating a system for coupling a medical device to an epicardial site using a delivery catheter according to the present invention. As illustrated in FIGS. 6 and 7, a system 200 for coupling a medical device to an epicardial site according to the present invention includes positioning delivery catheter 40 at a location within coronary sinus 4, near a junction of coronary sinus 4 with great cardiac vein 9 and posterior-lateral vein 11, where distal tip portion 360 of delivery catheter shaft 350 is directed toward a target site on an epicardial surface 70 of heart 6. Delivery catheter 40 according to system 200 of the present invention helps to insure that once positioned within coronary sinus 4, distal tip portion 360 of catheter 40 extends inward toward the epicardial surface 70 of heart 6, rather than outward away from epicardial surface, i.e., distal tip portion 360 extends toward a portion of coronary sinus 4 that is directly adjacent to epicardial surface 70. In particular, as illustrated in FIG. 7, orientation curve 50 and distal curve 61, 61a, 61b direct distal tip portion 360 toward epicardial surface 70 so that piercing tip 251 of fixation element 25 becomes fixedly engaged along epicardial surface 70 along heart wall 71 adjacent coronary sinus 4, rather than to an opposite wall 72 of surrounding CS 4 that is not directly adjacent heart wall 71, as fixation element 25 is extended outward from distal tip portion 360 and rotated to fixedly engage fixation element 25 within epicardial surface 70 and heart wall 70 of heart 6.

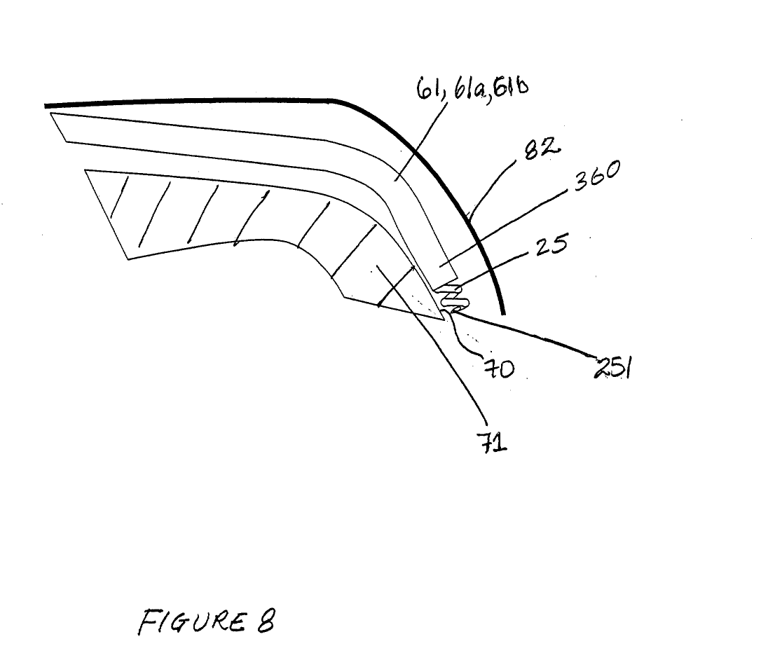

FIG. 8 is a sectional view taken along section line B-B of FIG. 6 illustrating a system for coupling a medical device to an epicardial site using a delivery catheter according to an alternate embodiment of the present invention. As illustrated in FIGS. 6 and 8, system 200 for coupling a medical device to an epicardial site according to an alternate embodiment of the present invention includes positioning delivery catheter 40 along a location within coronary sinus 4, with distal tip portion 360 of shaft 350 advanced into great cardiac vein 9. In the same way, according to the present invention, as catheter 40 is advanced along great cardiac vein 9, orientation curve 50 and distal curve 61,61a, 61b direct distal tip 370 along a course of great cardiac vein 9 so that distal tip portion 360 of catheter 40 extends inward toward epicardial surface 70 of heart 6 directly adjacent to great cardiac vein 9, rather than outward toward an opposite wall 82 of surrounding great cardiac vein 9 that is not directly adjacent heart wall 71, as fixation element 25 is extended outward from distal tip portion 360 of lead 20 and rotated to fixedly engage fixation element 25 within epicardial surface 70 and heart wall 70 of heart 6.

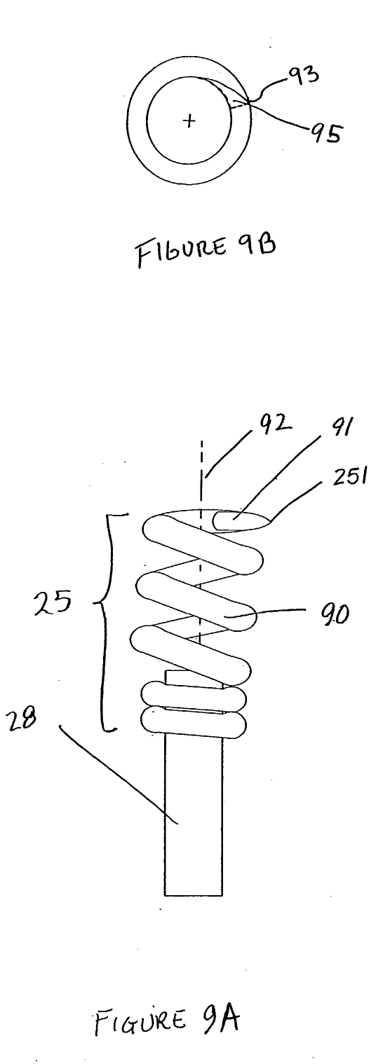

FIG. 9A is a side view of a helix fixation element in a system for coupling a medical device to an epicardial site using a delivery catheter according to the present invention. FIG. 9B is a top view of the helix fixation element of FIG. 9A. As illustrated in FIGS. 9A and 9B, fixation element 25 includes a helically wound wire 90 coupled to inner member 28 using a weld or a crimp or other coupling element well known in the art. According to the present invention, a surface 91 that creates piercing tip 251 is approximately flat and facing in the direction a central axis 92 of fixation element 25 and faces inward toward axis 92. According to the present invention, surface 90 is formed by grinding off an inner portion 95 of wire end 93 (shown with dashed lines). As fixation element 25 is directed outward from delivery catheter distal tip portion 360, constrained within a coronary vein, an oblique angle is formed between central axis 92 and an epicardial surface at a target site, as illustrated in FIGS. 7 and 8, so that orientation of surface 90, according to the present invention, accommodates engagement of piercing distal tip 251 against epicardial surface 70 as fixation element 25 is rotated.

Once fixation element 25 is rotated to become engaged within epicardial surface 70 along a target site, delivery catheter 40 is removed from venous system, over lead body 21. Subsequently, proximal portion 22 of pacing lead 20 is coupled to a medical device so that connector pin 23 (and ring 32, if pacing lead 20 is bipolar) comes into electrical contact with medical device, coupling medical device to an epicardial site via pacing lead 20.

Although the invention has been described in detail with particular reference to preferred embodiments and applications, those skilled in the art will recognize that variations and modifications can be effected within the scope of the following claims. For instance, the system described herein may include a therapy delivery device other than a pacing lead and a delivery catheter may be directed to an epicardial surface in approaches other than transvenous, such as transthoracic.

We claim:

1. A device for delivering an implantable medical device to a target site in a heart along a predetermined pathway, comprising:
a generally straight first portion extending from a proximal end to a distal end;
a curved second portion extending from the distal end of the first portion to a distal end of the device, the curved second portion including a first curve portion formed in a first plane and a second curved portion formed in a second plane substantially orthogonal to the first plane to direct the implantable medical device toward an epicardial surface of the heart directly adjacent to the predetermined pathway.

2. The device of claim 1, wherein the predetermined pathway is the coronary sinus of the heart.

3. The device of claim 1, wherein the curved second portion includes a first section, a second section and a third section extending between the first section and the second section, and wherein the first section is positioned at a first angle from the third section in the first plane and the second section is positioned at a second angle from the generally straight first portion in the second plane, the first angle being approximately equal to 90 degrees and the second angle being approximately equal to 30 degrees.

4. The device of claim 1, wherein the curved second portion has a radius of curvature between approximately 1.5 inches and 2.0 inches.

5. The device of claim 3, wherein an axial length of the generally straight first portion is between approximately 10 inches and 15 inches, an axial length of the first section is between approximately 3 inches and 5 inches, an axial length of the third section is between approximately 1 inch and 2 inches, and an axial length of the second section is between approximately 0.15 inches and 0.2 inches.

6. The device of claim 5, wherein the second section includes a second section curve and an axial length of the second section curve is between approximately 0.4 inches and 0.5 inches.

7. The device of claim 5, wherein the second section includes a second section curve having a radius of curvature between approximately 0.35 inches and 0.45 inches, and wherein an outer diameter of the generally straight first portion is between approximately 0.06 inches and 0.07 inches.

8. The device of claim 5, wherein the second section includes a second section curve having a radius of curvature between approximately 0.45 inches and 0.60 inches, and wherein an outer diameter of the generally straight first portion is between approximately 0.05 inches and 0.06 inches.

9. The device of claim 1, wherein a central axis of the third section is at an angle of approximately 90 degrees from a central axis of the generally straight first portion, and a central axis of second section is at an angle of approximately 30 degrees from the central axis of the generally straight first portion.

10. A device for delivering an implantable medical device to a target site in a heart along a predetermined pathway through the coronary sinus, comprising:
a generally straight first portion extending from a proximal end to a distal end;
a curved second portion extending from the distal end of the first portion to a distal end of the device, the curved second portion including a first curve portion formed in a first plane and a second curved portion formed in a second plane substantially orthogonal to the first plane to direct the implantable medical device toward an epicardial surface of the heart directly adjacent to the predetermined pathway, wherein the curved second portion includes a first section, a second section and a third section extending between the first section and the second section, and wherein the first section is positioned at a first angle from the third section in the first plane and the second section is positioned at a second angle from the generally straight first portion in the second plane, the first angle being approximately equal to 90 degrees and the second angle being approximately equal to 30 degrees, and wherein the curved second portion has a radius of curvature between approximately 1.5 inches and 2.0 inches.

11. The device of claim 10, wherein an axial length of the generally straight first portion is between approximately 10 inches and 15 inches, an axial length of the first section is between approximately 3 inches and 5 inches, an axial length of the third section is between approximately 1 inch and 2 inches, and an axial length of the second section is between approximately 0.15 inches and 0.2 inches.

12. The device of claim 10, wherein the second section includes a second section curve and an axial length of the second section curve is between approximately 0.4 inches and 0.5 inches.

13. The device of claim 10, wherein the second section includes a second section curve having a radius of curvature between approximately 0.35 inches and 0.45 inches, and wherein an outer diameter of the generally straight first portion is between approximately 0.06 inches and 0.07 inches.

14. The device of claim 10, wherein the second section includes a second section curve having a radius of curvature between approximately 0.45 inches and 0.60 inches, and wherein an outer diameter of the generally straight first portion is between approximately 0.05 inches and 0.06 inches.

15. A system for delivering an implantable medical device to a target site in a heart along a predetermined pathway through the coronary sinus, comprising:
a delivery catheter having a generally straight first portion extending from a first proximal end to a first distal end and a curved second portion extending from the first distal end to a distal end of the delivery catheter; and
a therapy delivery device, slideably receivable within the delivery catheter, extending from a second proximal end to a second distal end, wherein the curved second portion includes a first curve portion formed in a first plane and a second curved portion formed in a second plane substantially orthogonal to the first plane to direct the therapy delivery device outward from the distal end of the delivery catheter toward an epicardial surface of the heart directly adjacent to the coronary sinus.

16. The system of claim 15, wherein the curved second portion includes a first section, a second section and a third section extending between the first section and the second section, and wherein the first section is positioned at a first angle from the third section in the first plane and the second section is positioned at a second angle from the generally straight first portion in the second plane, the first angle being approximately equal to 90 degrees and the second angle being approximately equal to 30 degrees.

17. The system of claim 15, wherein the curved second portion has a radius of curvature between approximately 1.5 inches and 2.0 inches.

18. The system of claim 16, wherein an axial length of the generally straight first portion is between approximately 10 inches and 15 inches, an axial length of the first section is between approximately 3 inches and 5 inches, an axial length of the third section is between approximately 1 inch and 2 inches, and an axial length of the second section is between approximately 0.15 inches and 0.2 inches.

19. The system of claim 18, wherein the second section includes a second section curve and an axial length of the second section curve is between approximately 0.4 inches and 0.5 inches.

20. The system of claim 18, wherein the second section includes a second section curve having a radius of curvature between approximately 0.35 inches and 0.45 inches, and wherein an outer diameter of the generally straight first portion is between approximately 0.06 inches and 0.07 inches.

21. The system of claim 18, wherein the second section includes a second section curve having a radius of curvature between approximately 0.45 inches and 0.60 inches, and wherein an outer diameter of the generally straight first portion is between approximately 0.05 inches and 0.06 inches.

22. The system of claim 15, wherein a central axis of the third section is at an angle of approximately 90 degrees from a central axis of the generally straight first portion, and a central axis of second section is at an angle of approximately 30 degrees from the central axis of the generally straight first portion.

23. The system of claim 15, further comprising a fixation element coupled to the distal end of the therapy delivery device fixedly engaging the therapy delivery device to the epicardial surface, the fixation element having a tip portion approximately flat along a direction of a central axis of the fixation element and facing inward toward the central axis.
